(19) United States
(12) Patent Application Publication
(10) Pub. No.: US 2019/0365681 A1
(43) Pub. Date: Dec. 5, 2019

(54) TREATMENT OF CANCER WITH COMBINATIONS OF RXR AGONISTS AND THYROID HORMONES

(71) Applicant: Io Therapeutics, Inc., Santa Ana, CA (US)

(72) Inventors: Roshantha A. Chandraratna, San Juan Capistrano, CA (US); Martin E. Sanders, Seattle, WA (US)

(21) Appl. No.: 15/769,551

(22) PCT Filed: Oct. 31, 2016
(86) PCT No.: PCT/US2016/059779

§ 371 Date: Apr. 19, 2018

Related U.S. Application Data
(60) Provisional application No. 62/249,219, filed on Oct. 31, 2015.

(51) Int. Cl. A61K 31/192; A61K 31/216; A61K 31/4418; A61K 31/135; A61K 9/00; A61K 31/517

(52) U.S. Cl. CPC A61K 31/192; A61K 31/216; A61K 31/4418; A61K 31/517; A61K 9/0043; A61K 9/0053; A61K 9/0019; A61K 31/135

(57) ABSTRACT

The present specification provides methods of treating cancer with a combination of a RXR agonist and a thyroid hormone.

### RELATED APPLICATIONS

This application is a 35 U.S.C. 371 national phase entry of PCT/US2016/059779, filed Oct. 31, 2016, which claims priority to U.S. Provisional Patent Application No. 62/249,219, filed on Oct. 31, 2015. The entire content of each of these applications is herein incorporated by reference.

### FIELD

The present disclosure is directed to methods of treating cancer associated with the biochemical functions modulated by Retinoid X Receptors (RXR) using RXR agonists in combination with a thyroid hormone.

### BACKGROUND

Compounds which have retinoid-like biological activity are well known in the art and are described in numerous United States patents including, but not limited to, U.S. Pat. Nos. 5,466,861; 5,675,033; and 5,917,082, all of which are herein incorporated by reference. Preclinical studies with rexinoids, which are agonists of RXRs, suggest that selective activation of retinoid X receptors (RXR), which modulate functions associated with differentiation, inhibition of cell growth, apoptosis and metastasis, may be useful in treating a variety of diseases associated with the biochemical functions modulated by RXR.

For example, bexarotene (TARGRETIN), which is a RXR agonist with retinoic acid receptor (RAR) agonist activity as well, was approved by the U.S. Food and Drug Administration for the treatment, both oral and topical, of cutaneous manifestations of cutaneous T-cell lymphoma in patients who are refractory to at least one prior systemic therapy. Further, recent clinical studies that were conducted using bexarotene suggest that there is potential for RXR agonists in the treatment of non-small cell lung cancer (NSCLC). Encouraging results were obtained with bexarotene in several Phase II studies in NSCLC. However, the pivotal Phase III clinical studies with bexarotene in NSCLC patients did not show increased survival of the subjects relative to control. Therefore, there is a need for new technologies that increase the efficacy of RXR agonists when used to treat cancers, especially for solid tumors such as NSCLC.

### SUMMARY

The activation of Retinoic Acid Receptors (RAR) by non-selective Retinoic X Receptor (RXR) agonists decreases the efficacy of the RXR agonists as anti-cancer agents. As such, the efficacy of RXR agonists in cancer can be improved by administering the RXR agonist at a dose which activates RXR but which activates RAR minimally or not at all. It is now proposed that a RXR agonist at a dose which specifically activates only RXRs gives optimal anti-cancer activity when combined with administration of a thyroid hormone. Based on this proposal, novel methods of treating a patient with cancer are disclosed herein.

Thus, disclosed herein, are methods of treating cancer comprising administering to a patient in need of such treatment a RXR agonist at a level below the RAR activating threshold and at or above the RXR effective dose. In another embodiment provided are methods of treating cancer comprising dosing a patient in need of such treatment with an effective concentration of a RXR agonist in combination with an effective dose of a thyroid hormone. In another embodiment are methods of treating a cancer patient with increasing doses of a RXR agonist until a dose is found which is a RXR effective dose but is below the RAR activating threshold and using this dose of a RXR agonist in combination with an effective dose of a thyroid hormone to treat the cancer patient.

In some embodiments, the RXR agonist may have the structure of Formula II

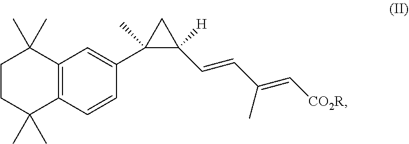

wherein R is H or lower alkyl of 1 to 6 carbons.

In other embodiments, the RXR agonist may be 3,7-dimethyl-6(S),7(S)-methano,7-[1,1,4,4-tetramethyl-1,2,3,4-tetrahydronaphth-7-yl]2(E),4(E) heptadienoic ethyl ester, 3,7-dimethyl-6(S),7(S)-methano,7-[1,1,4,4-tetramethyl-1,2,3,4-tetrahydronaphth-7-yl]2(E),4(E) heptadienoic acid, bexarotene, LG268, or combinations thereof. In some embodiments, the therapeutically effective amount of the RXR agonist may be about 0.001 mg/day to about 1000 mg/day, about 10 mg/day to about 1000 mg/day, about 1 mg/day to about 100 mg/day, or any range bound by these values.

In some embodiments, the method may comprise administering to an individual in need thereof a therapeutically effective amount of 3,7-dimethyl-6(S),7(S)-methano,7-[1,1,4,4-tetramethyl-1,2,3,4-tetrahydronaphth-7-yl]2(E),4(E) heptadienoic acid, and a therapeutically acceptable amount of thyroxine; and wherein administration of the combination reduces the size of at least one tumor in the individual.

In some embodiments, the thyroid hormone may be thyroxine. In other embodiments, the therapeutically effective amount of thyroxine may be about 12.5 μg/day to about 250 μg/day.

In some embodiments, the RXR agonist may be administered by nasal or oral administration. In other embodiments, the thyroxine may be administered orally or subcutaneously. In some embodiments, the RXR agonist and thyroxine may be both administered by nasal administration. In still other embodiments, the RXR agonist and the thyroxine may be both administered substantially simultaneously. In other embodiments, the RXR agonist and thyroxine may be administered on different schedules.

In some embodiments, the method may treat a cancer. In other embodiments, the cancer may be lung cancer, prostate cancer, breast cancer, pancreatic cancer, or other solid tumor cancers.

In some embodiments, the method of treating cancer with a RXR agonist and thyroid hormone reduces the size of a tumor by about 5% to about 100%.

In some embodiments, the method of treating cancer with a RXR agonist and thyroid hormone increases a 1-year survival rate by about 5% to about 100%.

In some embodiments, the method of treating cancer with a RXR agonist and thyroid hormone increases a 5-year survival rate by about 5% to about 100%.

In some embodiments, the method of treating cancer with a RXR agonist and thyroid hormone increases a 10-year survival rate by about 5% to about 100%.

In other embodiments, further comprising administration of another anti-cancer agent. In some embodiments, the anti-cancer agent may be TARCEVA (erlotinib), TAXOL (paclitaxel), TAXOTERE (docetaxel), and combinations thereof.

### BRIEF DESCRIPTION OF THE DRAWINGS

FIG. 1 shows RXR agonist activation of transcription from RXRα, RXRβ, RXRγ, RARα, RARβ, and RARγ using transactivation assays.

FIGS. 2A-D shows that IRX4204 selectively activates RXR-Nurr1 heterodimers. Transactivation assay of IRX4204 (194204, Formula III) for farnesoid X receptor FXR (FIG. 2A); for liver X receptors LXRα and LXRβ (FIG. 2B); for peroxisome proliferator-activated receptor PPARγ (FIG. 2C); and for Nurr1 receptor in the presence or absence of RXR (FIG. 2D).

FIGS. 3A-C show the anti-tumor effects of IRX4204 on nude mice xenografted with human H292 non-small cell lung cancer (NSCLC) tumors. Nude mice were randomized into 4 groups of 10 animals each based on body weight and xenografted subcutaneously in the right flank with H292 cells (2×10⁶ cells). Drug treatment was started immediately after xenografting and continued for 35 days (5 animals of each group) or 55 days (remaining 5 animals). The animals were treated with vehicle (VEH), paclitaxol (TAXOL) 5 mg/kg/week, once a week, i.p., IRX4204 10 mg/kg/day, 5 days a week, by oral gavage, or IRX4204+TAXOL. Tumor sizes measured periodically for 35 days (FIG. 3A). Animals #1-5 of each group were sacrificed after 35 days of treatment and cross-sectional areas of gastrocnemius muscles were determined (FIG. 3C). The body weights and overall appearance of animals #6 through #10 from each group were followed for an extended period (FIG. 3B).

FIG. 4 shows a graph of duration of tumor non-progression when patients were administered IRX4204.

### DETAILED DESCRIPTION

Preclinical studies with rexinoids suggest that selective activation of Retinoid X Receptors (RXR), which modulate functions associated with differentiation, inhibition of cell growth, apoptosis and metastasis, may be useful in treating a variety of diseases associated with the biochemical functions modulated by RXR.

The RARs and RXRs and their cognate ligands function by distinct mechanisms. The term “RAR” as used herein refers to one or more of RARα, RARβ, or RARγ. The term “RXR” as used herein refers to one or more of RXRα, RXRβ, or RXRγ. A RAR biomarker is a distinctive biological, biochemical or biologically derived indicator that signifies patient RAR activity. RAR biomarkers include, but are not limited to, CYP26 levels, CRBPI levels, and the like, and combinations thereof.

RAR activation threshold means one or more of a CYP26 level which is 25% increased over baseline and a CRBPI level 25% increased over baseline. The RARs form heterodimers with RXRs and these RAR/RXR heterodimers bind to specific response elements in the promoter regions of target genes. The binding of RAR agonists to the RAR receptor of the heterodimer results in activation of transcription of target genes leading to retinoid effects. On the other hand, RXR agonists do not activate RAR/RXR heterodimers. RXR heterodimer complexes like RAR/RXR can be referred to as non-permissive RXR heterodimers as activation of transcription due to ligand-binding occurs only at the non-RXR protein (e.g., RAR); activation of transcription does not occur due to ligand binding at the RXR. RXRs also interact with nuclear receptors other than RARs and RXR agonists may elicit some of its biological effects by binding to such RXR/receptor complexes.

These RXR/receptor complexes can be referred to as permissive RXR heterodimers as activation of transcription due to ligand-binding could occur at the RXR, the other receptor, or both receptors. Examples of permissive RXR heterodimers include, without limitation, peroxisome proliferator activated receptor/RXR (PPAR/RXR), farnesyl X receptor/RXR (FXR/RXR), nuclear receptor related-1 protein (Nurr1/RXR) and liver X receptor/RXR (LXR/RXR). Alternately, RXRs may form RXR/RXR homodimers which can be activated by RXR agonists leading to rexinoid effects. Also, RXRs interact with proteins other than nuclear receptors and ligand binding to an RXR within such protein complexes can also lead to rexinoid effects. Due to these differences in mechanisms of action, RXR agonists and RAR agonists elicit distinct biological outcomes and even in the instances where they mediate similar biological effects, they do so by different mechanisms. Moreover, the unwanted side effects of retinoids, such as pro-inflammatory responses or mucocutaneous toxicity, are mediated by activation of one or more of the RAR receptor subtypes. Stated another way, biological effects mediated via RXR pathways would not induce pro-inflammatory responses, and thus, would not result in unwanted side effects.

Thus, aspects of the present specification provide, in part, a RXR agonist. As used herein, the term “RXR agonist”, is synonymous with “selective RXR agonist” and refers to a compound that selectively binds to one or more RXR receptors like a RXRα, a RXRβ, or a RXRγ in a manner that elicits gene transcription via an RXR response element. As used herein, the term “selectively binds,” when made in reference to a RXR agonist, refers to the discriminatory binding of a RXR agonist to the indicated target receptor like a RXRα, a RXRβ, or a RXRγ such that the RXR agonist does not substantially bind with non-target receptors like a RARα, a RARβ or a RARγ. In some embodiments, the term “RXR agonist” includes esters of RXR agonist.

In one embodiment, the selective RXR agonist does not activate to any appreciable degree the permissive heterodimers PPAR/RXR, FXR/RXR, and LXR/RXR. In another embodiment, the RXR agonist, activates the permissive heterodimer Nurr1/RXR. One example of such a selective RXR agonist is 3,7-dimethyl-6(S),7(S)-methano,7-[1,1,4,4-tetramethyl-1,2,3,4-tetrahydronaphth-7-yl]2(E),4(E) heptadienoic acid (IRX4204) disclosed herein, the structure of which is shown in Formula III. In other aspects of this embodiment, the RXR agonists, activates the permissive heterodimers PPAR/RXR, FXR/RXR, or LXR/RXR by 1% or less, 2% or less, 3% or less, 4% or less, 5% or less, 6% or less, 7% or less, 8% or less, 9% or less, or 10% or less relative to the ability of activating agonists to the non-RXR receptor to activate the same permissive heterodimer. Examples of RXR agonists, which activates one or more of PPAR/RXR, FXR/RXR, or LXR/RXR include, LGD1069 (bexarotene) and LGD268.

IRX4204, like some other RXR ligands, does not activate non-permissive heterodimers such as RAR/RXR. However, IRX4204, is unique in that it specifically activates the Nurr1/RXR heterodimer and does not activate other permissive RXR heterodimers such as PPAR/RXR, FXR/RXR, and LXR/RXR. Other RXR ligands generally activate these permissive RXR heterodimers. Thus, all RXR ligands cannot be classified as belonging to one class. IRX4204 belongs to a unique class of RXR ligands which specifically activate RXR homodimers and only one of the permissive RXR heterodimers, namely the Nurr1/RXR heterodimer.

Binding specificity is the ability of a RXR agonist to discriminate between a RXR receptor and a receptor that does not contain its binding site, such as a RAR receptor.

Also disclosed herein are esters of RXR agonists. An ester may be derived from a carboxylic acid of C1, or an ester may be derived from a carboxylic acid functional group on another part of the molecule, such as on a phenyl ring. While not intending to be limiting, an ester may be an alkyl ester, an aryl ester, or a heteroaryl ester. The term alkyl has the meaning generally understood by those skilled in the art and refers to linear, branched, or cyclic alkyl moieties. C₁₋₆ alkyl esters are particularly useful, where alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, etc.

Thus, disclosed herein are RXR agonists, or esters thereof, having the structure of formula I:

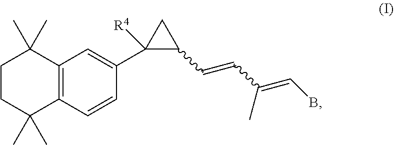

where R⁴ is lower alkyl of 1 to 6 carbons; B is —COOR^{S} where R⁸ is lower alkyl of 1 to 6 carbons, the configuration about the cyclopropane ring is cis, and the configuration about the double bonds in the pentadienoic acid or ester chain attached to the cyclopropane ring is trans in each of the double bonds.

In an exemplary embodiment, an ester of a RXR agonist is a compound having the structure of formula II:

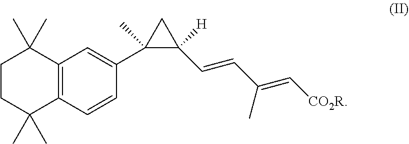

wherein R is H or lower alkyl of 1 to 6 carbons.

In a further exemplary embodiment, a selective RXR agonist may be 3,7-dimethyl-6(S),7(S)-methano,7-[1,1,4,4-tetramethyl-1,2,3,4-tetrahydronaphth-7-yl]2(E),4(E) heptadienoic acid or esters thereof, and has the structure of formula III:

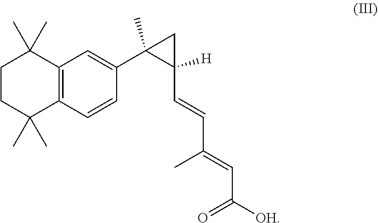 [SMILES: CC(/C=C/[C@@H]1C[C@]1(C)c1ccc2c(c1)C(C)(C)CCC2(C)C)=C\C(=O)O]

In certain embodiments, the RXR agonist may be bexarotene (TARGRETIN, 4-[1-(3,5,5,8,8-pentamethyl-6,7-dihydronaphthalen-2-yl)ethenyl]benzoic acid, LGD1069, Mylan Pharmaceuticals, Inc.), or esters thereof, and has the structure of formula IV:

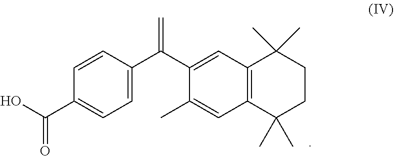 [SMILES: C=C(c1ccc(C(=O)O)cc1)c1cc2c(cc1C)C(C)(C)CCC2(C)C]

In other embodiments, the RXR agonist may be LG268 (LG100268, LGD268, 2-[1-(3,5,5,8,8-pentamethyl-5,6,7,8-tetrahydro-2-naphthyl)cyclopropyl]pyridine-5-carboxylic acid), or esters thereof and has the structure of formula V:

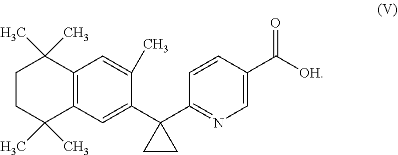 [SMILES: Cc1cc2c(cc1C1(c3ccc(C(=O)O)cn3)CC1)C(C)(C)CCC2(C)C]

Pharmaceutically acceptable salts of RXR agonists, or esters thereof, can also be used in the disclosed method. Compounds disclosed herein which possess a sufficiently acidic, a sufficiently basic, or both functional groups, and accordingly can react with any of a number of organic or inorganic bases, and inorganic and organic acids, to form a salt.

Administration of RXR agonists, or esters thereof, may lead to the suppression of serum thyroid hormones and possibly to hypothyroidism and related conditions. In some embodiments, a thyroid hormone may be used in combination with the RXR agonists, or esters thereof. As used herein, the term “thyroid hormone” refers to thyroxine and triiodothyronine. Thyroxine (thyroid hormone T₄, levothyroxine sodium) is a tyrosine-based hormone produced by the thyroid gland and is primarily responsible for regulation of metabolism. Thyroxine is a prohormone for triiodothyronine (T₃). RXR agonists are known to suppress thyroid function. However supplementation of RXR agonist therapy with thyroid hormones has not been utilized therapeutically to enhance the anti-cancer effects of a RXR agonist.

Aspects of the present specification provide, in part, a composition comprising a RXR agonist, or ester or other derivative thereof, and compositions comprising a RXR agonist, or ester or other derivative thereof, and a thyroid hormone. Exemplary RXR agonists are IRX4204, bexarotene, and LG268. Exemplary esters of RXR agonists are IRX4204 ethyl ester (IRX4204EE), an ester of bexarotene, and an ester of LG268.

Aspects of the methods of the present disclosure include, in part, treatment of a mammal. A mammal includes a human, and a human can be a patient. Other aspects of the present disclosure provide, in part, an individual. An individual includes a mammal and a human, and a human can be a patient.

In some embodiments, the RXR agonists, or esters thereof, may be optionally administered in the disclosed methods in combination with a second anti-cancer agent. Suitable anti-cancer agents include cytotoxic drugs, including, but not limited to, TARCEVA (erlotinib), TAXOL (paclitaxel), TAXOTERE (docetaxel), and the like and mixtures thereof. Additional anti-cancer agents include ADRIAMYCIN, dactinomycin, bleomycin, vinblastine, cisplatin, acivicin, aclarubicin, acodazole, acronine, adozelesin, aldesleukin, altretamine, ambomycin, ametantrone acetate; aminoglutethimide, amsacrine, anastrozole; anthramycin, asparaginase, asperlin, azacitidine, azetepa, azotomycin, batimastat, benzodepa, bicalutamide, bisantrene; bisnafide dimesylate, bizelesin, bleomycin sulfate, brequinar sodium, bropirimine, busulfan, cactinomycin, calusterone, caracemide, carbetimer, carboplatin, carmustine, carubicin, carzelesin, cedefingol, chlorambucil, cirolemycin, cladribine, crisnatol mesylate, cyclophosphamide, cytarabine, dacarbazine, daunorubicin, decitabine, dexormaplatin; dezaguanine, dezaguanine mesylate, diaziquone, doxorubicin, droloxifene, dromostanolone propionate, duazomycin, edatrexate, eflornithine, elsamitrucin, enloplatin, enpromate, epipropidine, epirubicin, erbulozole, esorubicin, estramustine, etanidazole, etoposide, etoposide, etoprine, fadrozole, fazarabine, fenretinide, floxuridine, fludarabine phosphate, fluorouracil, fluorocitabine, fosquidone, fostriecin sodium, gemcitabine, hydroxyurea, idarubicin, ifosfamide, ilmofosine, interleukin 11 (including recombinant interleukin 11, or rIL2), interferon alfa-2a, interferon alfa-2b, interferon alfa-n1, interferon alfa-n3, interferon beta-1a, interferon gamma-1b, iproplatin, irinotecan, lanreotide, letrozole, leuprolide, liarozole, lometrexol, lomustine, losoxantrone, masoprocol, maytansine, mechlorethamine, megestrol, melengestrol, melphalan, menogaril; mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nocodazole, nogalamycin, ormaplatin, oxisuran, pegaspargase, peliomycin, pentamustine, peplomycin, perfosfamide, pipobroman, piposulfan, piroxantrone, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tecogalan, tegafur, teloxantrone, temoporfin, teniposide, teroxirone, testolactone, thiamiprine, thioguanine, thiotepa, tiazofurin, tirapazamine, toremifene, trestolone, triciribine, trimetrexate; trimetrexate glucuronate, triptorelin, tubulozole, uracil mustard, uredepa, vapreotide, verteporfin, vinblastine, vincristine, vindesine, vinepidine, vinglycinate, vinleurosine, vinorelbine, vinrosidine, vinzolidine, vorozole, zeniplatin, zinostatin, and zorubicin. Additional anti-cancer drugs may be 5-fluorouracil and leucovorin. Platinum-based drugs may include, but are not limited to, carboplatin, cisplatin, and the like, and mixtures thereof.

In some embodiments, the anti-cancer agent is TARCEVA (erlotinib).

In some embodiments, the second anti-cancer agent may include, but are not limited to, a platinum-based compound, a cytotoxic drug, a receptor tyrosine kinase inhibitor, and combinations thereof.

RXR agonists, or esters thereof, disclosed herein, or a composition comprising a RXR agonist, or ester thereof, or a combination of a RXR agonist, or ester thereof, and a thyroid hormone, such as thyroxine, is generally administered to an individual as a pharmaceutical composition.

Pharmaceutical compositions may be prepared by combining a therapeutically effective amount of at least one RXR agonist, as an active ingredient, with conventional acceptable pharmaceutical excipients, and by preparation of unit dosage forms suitable for therapeutic use. As used herein, the term “pharmaceutical composition” refers to a therapeutically effective concentration of an active compound, such as any of the compounds disclosed herein. Preferably, the pharmaceutical composition does not produce an adverse, allergic, or other untoward or unwanted reaction when administered to an individual. A pharmaceutical composition disclosed herein is useful for medical and veterinary applications. A pharmaceutical composition may be administered to an individual alone, or in combination with other supplementary active compounds, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir, or any other dosage form suitable for administration.

A pharmaceutical composition produced using the methods disclosed herein may be a liquid formulation, semi-solid formulation, or a solid formulation. A formulation disclosed herein can be produced in a manner to form one phase, such as, but not limited to an oil or a solid. Alternatively, a formulation disclosed herein can be produced in a manner to form two phases, such as an emulsion. A pharmaceutical composition disclosed herein intended for such administration may be prepared according to any method known to the art for the manufacture of pharmaceutical compositions.

Liquid formulations suitable for parenteral injection or for nasal sprays may comprise physiologically acceptable sterile aqueous or nonaqueous solutions, dispersions, suspensions or emulsions and sterile powders for reconstitution into sterile injectable solutions or dispersions. Formulations suitable for nasal administration may comprise physiologically acceptable sterile aqueous or nonaqueous solutions, dispersions, suspensions or emulsions. Examples of suitable aqueous and nonaqueous carriers, diluents, solvents or vehicles include, but are not limited to, water, ethanol, polyols (propylene glycol, polyethyleneglycol (PEG), glycerol, and the like), suitable mixtures thereof, vegetable oils (such as olive oil or peanut oil) and injectable organic esters such as ethyl oleate. Proper fluidity can be maintained, for example, by the use of a coating such as lecithin, by the maintenance of the required particle size in the case of dispersions and by the use of surfactants.

Aqueous suspensions may include pharmaceutically acceptable excipients such as, but not limited to, a) suspending agents, as for example, sodium carboxymethyl cellulose, methylcellulose, hydroxypropylmethylcellulose, sodium alginate, polyvinylpyrrolidone, gum tragacanth and gum acacia; b) dispersing or wetting agents, as for naturally occurring phosphatide or lecithin, or condensation products of an alkylene oxide with fatty acids, such as, but not limited to, polyoxyethylene stearate, or condensation products of ethylene oxide with long chain aliphatic alcohols, such as, but not limited to, heptadecaethylene-oxycetanol, or condensation products of ethylene oxide with partial esters derived from fatty acids and a hexitol, such as polyoxyethylene sorbitol monoleate or condensation products of ethylene oxide with partial esters derived from fatty acids and hexitol anhydrides, such as, but not limited to, polyoxyethylene sorbitan monoleate. The aqueous suspensions can also contain one or more preservatives, ethyl- or -n-propyl-p-hydroxy benzoate, one or more coloring agents, one or more flavoring agents and one or more sweetening agents, such as, but not limited to, sucrose, saccharin or sodium or calcium cyclamate.

Pharmaceutical formulations suitable for administration by inhalation include fine particle dusts or mists, which may be generated by means of various types of metered, dose pressurized aerosols, nebulizers, or insufflators.

Semi-solid formulations suitable for topical administration include, without limitation, ointments, creams, salves, and gels. In such solid formulations, the active compound may be admixed with at least one inert customary excipient (or carrier) such as, but not limited to, a lipid and/or polyethylene glycol.

Solid formulations suitable for oral administration include capsules, tablets, pills, powders and granules. In such solid formulations, the active compound may be admixed with at least one inert customary excipient (or carrier) such as, but not limited to, sodium citrate or dicalcium phosphate or (a) fillers or extenders, for example but not limited to, starches, lactose, sucrose, glucose, mannitol and silicic acid, (b) binders, for example but not limited to, carboxymethylcellulose, alignates, gelatin, polyvinylpyrrolidone, sucrose and acacia, (c) humectants, for example, but not limited to, glycerol, (d) disintegrating agents, for example, but not limited to, agar-agar, calcium carbonate, potato or tapioca starch, alginic acid, certain complex silicates and sodium carbonate, (e) solution retarders, for example, but not limited to, paraffin, (f) absorption accelerators, for example, but not limited to, quaternary ammonium compounds, (g) wetting agents, for example, but not limited to, cetyl alcohol and glycerol monostearate, (h) adsorbents, for example, but not limited to, kaolin and bentonite, and (i) lubricants, for example, but not limited to, talc, calcium stearate, magnesium stearate, solid polyethylene glycols, sodium lauryl sulfate or mixtures thereof. In the case of capsules, tablets and pills, the dosage forms may also comprise buffering agents.

In liquid and semi-solid formulations, a concentration of a RXR agonist typically may be between about 50 mg/mL to about 1,000 mg/mL. In aspects of this embodiment, a therapeutically effective amount of a therapeutic compound disclosed herein may be from about 50 mg/mL to about 100 mg/mL, about 50 mg/mL to about 200 mg/mL, about 50 mg/mL to about 300 mg/mL, about 50 mg/mL to about 400 mg/mL, about 50 mg/mL to about 500 mg/mL, about 50 mg/mL to about 600 mg/mL, about 50 mg/mL to about 700 mg/mL, about 50 mg/mL to about 800 mg/mL, about 50 mg/mL to about 900 mg/mL, about 50 mg/mL to about 1,000 mg/mL, about 100 mg/mL to about 200 mg/mL, about 100 mg/mL to about 300 mg/mL, about 100 mg/mL to about 400 mg/mL, about 100 mg/mL to about 500 mg/mL, about 100 mg/mL to about 600 mg/mL, about 100 mg/mL to about 700 mg/mL, about 100 mg/mL to about 800 mg/mL, about 100 mg/mL to about 900 mg/mL, about 100 mg/mL to about 1,000 mg/mL, about 200 mg/mL to about 300 mg/mL, about 200 mg/mL to about 400 mg/mL, about 200 mg/mL to about 500 mg/mL, about 200 mg/mL to about 600 mg/mL, about 200 mg/mL to about 700 mg/mL, about 200 mg/mL to about 800 mg/mL, about 200 mg/mL to about 900 mg/mL, about 200 mg/mL to about 1,000 mg/mL, about 300 mg/mL to about 400 mg/mL, about 300 mg/mL to about 500 mg/mL, about 300 mg/mL to about 600 mg/mL, about 300 mg/mL to about 700 mg/mL, about 300 mg/mL to about 800 mg/mL, about 300 mg/mL to about 900 mg/mL, about 300 mg/mL to about 1,000 mg/mL, about 400 mg/mL to about 500 mg/mL, about 400 mg/mL to about 600 mg/mL, about 400 mg/mL to about 700 mg/mL, about 400 mg/mL to about 800 mg/mL, about 400 mg/mL to about 900 mg/mL, about 400 mg/mL to about 1,000 mg/mL, about 500 mg/mL to about 600 mg/mL, about 500 mg/mL to about 700 mg/mL, about 500 mg/mL to about 800 mg/mL, about 500 mg/mL to about 900 mg/mL, about 500 mg/mL to about 1,000 mg/mL, about 600 mg/mL to about 700 mg/mL, about 600 mg/mL to about 800 mg/mL, about 600 mg/mL to about 900 mg/mL, about 600 mg/mL to about 1,000 mg/mL, or any other range bound by these values.

In semi-solid and solid formulations, an amount of a RXR agonist may be between about 0.01% to about 45% by weight. In aspects of this embodiment, an amount of a therapeutic compound disclosed herein may be from about 0.1% to about 45% by weight, about 0.1% to about 40% by weight, about 0.1% to about 35% by weight, about 0.1% to about 30% by weight, about 0.1% to about 25% by weight, about 0.1% to about 20% by weight, about 0.1% to about 15% by weight, about 0.1% to about 10% by weight, about 0.1% to about 5% by weight, about 1% to about 45% by weight, about 1% to about 40% by weight, about 1% to about 35% by weight, about 1% to about 30% by weight, about 1% to about 25% by weight, about 1% to about 20% by weight, about 1% to about 15% by weight, about 1% to about 10% by weight, about 1% to about 5% by weight, about 5% to about 45% by weight, about 5% to about 40% by weight, about 5% to about 35% by weight, about 5% to about 30% by weight, about 5% to about 25% by weight, about 5% to about 20% by weight, about 5% to about 15% by weight, about 5% to about 10% by weight, about 10% to about 45% by weight, about 10% to about 40% by weight, about 10% to about 35% by weight, about 10% to about 30% by weight, about 10% to about 25% by weight, about 10% to about 20% by weight, about 10% to about 15% by weight, about 15% to about 45% by weight, about 15% to about 40% by weight, about 15% to about 35% by weight, about 15% to about 30% by weight, about 15% to about 25% by weight, about 15% to about 20% by weight, about 20% to about 45% by weight, about 20% to about 40% by weight, about 20% to about 35% by weight, about 20% to about 30% by weight, about 20% to about 25% by weight, about 25% to about 45% by weight, about 25% to about 40% by weight, about 25% to about 35% by weight, about 25% to about 30% by weight, or any other range bound by these values.

A pharmaceutical composition disclosed herein may optionally include a pharmaceutically acceptable carrier that facilitates processing of an active compound into pharmaceutically acceptable compositions. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. As used herein, the term “pharmacologically acceptable carrier” is synonymous with “pharmacological carrier” and refers to any carrier that has substantially no long term or permanent detrimental effect when administered and encompasses terms such as “pharmacologically acceptable vehicle, stabilizer, diluent, additive, auxiliary, or excipient.” Such a carrier generally is mixed with an active compound or permitted to dilute or enclose the active compound and can be a solid, semi-solid, or liquid agent. It is understood that the active compounds can be soluble or can be delivered as a suspension in the desired carrier or diluent.

Any of a variety of pharmaceutically acceptable carriers may be used including, without limitation, aqueous media such as water, saline, glycine, hyaluronic acid and the like; solid carriers such as starch, magnesium stearate, mannitol, sodium saccharin, talcum, cellulose, glucose, sucrose, lactose, trehalose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active compound, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003). These protocols are routine and any modifications are well within the scope of one skilled in the art and from the teaching herein.

A pharmaceutical composition disclosed herein may optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH may be used to prepare a pharmaceutical composition disclosed herein, provided that the resulting preparation is pharmaceutically acceptable. Such buffers include, without limitation, acetate buffers, borate buffers, citrate buffers, phosphate buffers, neutral buffered saline, and phosphate buffered saline. It is understood that acids or bases can be used to adjust the pH of a composition as needed.

Pharmaceutically acceptable antioxidants include, without limitation, sodium metabisulfite, sodium thiosulfate, acetylcysteine, butylated hydroxyanisole, and butylated hydroxytoluene. Useful preservatives may include, but not limited to, benzalkonium chloride, chlorobutanol, thimerosal, phenylmercuric acetate, phenylmercuric nitrate, a stabilized oxy chloro composition, sodium chlorite and chelants, DTPA or DTPA-bisamide, calcium DTPA, and CaNaDTPA-bisamide. Tonicity adjustors useful in a pharmaceutical composition may include, but are not limited to, salts such as sodium chloride, potassium chloride, mannitol or glycerin and other pharmaceutically acceptable tonicity adjustor. The pharmaceutical composition may be provided as a salt and can be formed with many acids, including, but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful herein.

The compounds disclosed herein, such as a combination of a RXR agonist and thyroxine, may also be incorporated into a drug delivery platform in order to achieve a controlled compound release profile over time. Such a drug delivery platform may comprise the combination disclosed herein dispersed within a polymer matrix, typically a biodegradable, bioerodible, and/or bioresorbable polymer matrix. As used herein, the term “polymer” refers to synthetic homo- or copolymers, naturally occurring homo- or copolymers, as well as synthetic modifications or derivatives thereof having a linear, branched or star structure. Copolymers can be arranged in any form, such as random, block, segmented, tapered blocks, graft, or triblock. Polymers are generally condensation polymers. Polymers can be further modified to enhance their mechanical or degradation properties by introducing cross-linking agents or changing the hydrophobicity of the side residues. If crosslinked, polymers are usually less than 5% crosslinked, usually less than 1% crosslinked.

Suitable polymers may include, but are not limited to, alginates, aliphatic polyesters, polyalkylene oxalates, polyamides, polyamidoesters, polyanhydrides, polycarbonates, polyesters, polyethylene glycol, polyhydroxyaliphatic carboxylic acids, polyorthoesters, polyoxaesters, polypeptides, polyphosphazenes, polysaccharides, and polyurethanes. The polymer usually comprises at least about 10% (w/w), at least about 20% (w/w), at least about 30% (w/w), at least about 40% (w/w), at least about 50% (w/w), at least about 60% (w/w), at least about 70% (w/w), at least about 80% (w/w), or at least about 90% (w/w) of the drug delivery platform. Examples of biodegradable, bioerodible, and/or bioresorbable polymers and methods useful to make a drug delivery platform are described in U.S. Pat. Nos. 4,756,911; 5,378,475; 7,048,946; and U.S. Patent Publication Nos. 2005/0181017; 2005/0244464; 2011/0008437; each of which is incorporated by reference for all it discloses regarding drug delivery.

In aspects of this embodiment, a polymer composing the matrix may be a polypeptide such as, but not limited to, silk fibroin, keratin, or collagen. In other aspects of this embodiment, a polymer composing the matrix may be a polysaccharide such as, but not limited to, cellulose, agarose, elastin, chitosan, chitin, or a glycosaminoglycan like chondroitin sulfate, dermatan sulfate, keratan sulfate, or hyaluronic acid. In yet other aspects of this embodiment, a polymer composing the matrix may be a polyester such as D-lactic acid, L-lactic acid, racemic lactic acid, glycolic acid, caprolactone, and combinations thereof.

One of ordinary skill in the art appreciates that the selection of a suitable polymer for forming a suitable disclosed drug delivery platform depends on several factors. The more relevant factors in the selection of the appropriate polymer(s), include, without limitation, compatibility of polymer with drug, desired release kinetics of drug, desired biodegradation kinetics of platform at implantation site, desired bioerodible kinetics of platform at implantation site, desired bioresorbable kinetics of platform at implantation site, in vivo mechanical performance of platform, processing temperatures, biocompatibility of platform, and patient tolerance. Other relevant factors that, to some extent, dictate the in vitro and in vivo behavior of the polymer include the chemical composition, spatial distribution of the constituents, the molecular weight of the polymer and the degree of crystallinity.

A drug delivery platform may include both a sustained release drug delivery platform and an extended release drug delivery platform. As used herein, the term “sustained release” refers to the release of a compound disclosed herein over a period of about seven days or more. As used herein, the term “extended release” refers to the release of a compound disclosed herein over a period of time of less than about seven days.

In aspects of this embodiment, a sustained release drug delivery platform may release a RXR agonist disclosed herein, or the combination a RXR agonist and a thyroid hormone, with substantially first order release kinetics over a period of about 7 days after administration, about 15 days after administration, about 30 days after administration, about 45 days after administration, about 60 days after administration, about 75 days after administration, or about 90 days after administration. In other aspects of this embodiment, a sustained release drug delivery platform releases a compound disclosed herein with substantially first order release kinetics over a period of at least 7 days after administration, at least 15 days after administration, at least 30 days after administration, at least 45 days after administration, at least 60 days after administration, at least 75 days after administration, or at least 90 days after administration.

In aspects of this embodiment, a drug delivery platform may release a RXR agonist disclosed herein, and a thyroid hormone, with substantially first order release kinetics over a period of about 1 day after administration, about 2 days after administration, about 3 days after administration, about 4 days after administration, about 5 days after administration, or about 6 days after administration. In other aspects of this embodiment, a drug delivery platform releases a compound disclosed herein with substantially first order release kinetics over a period of at most 1 day after administration, at most 2 days after administration, at most 3 days after administration, at most 4 days after administration, at most 5 days after administration, or at most 6 days after administration.

Aspects of the present disclosure include, in part, administering a RXR agonist, or a RXR agonist in combination with a thyroid hormone, such as thyroxine. As used herein, the term “administering” means any delivery mechanism that provides a compound, a composition, or a combination disclosed herein to an individual that potentially results in a clinically, therapeutically, or experimentally beneficial result.

Administration of a RXR agonist, in combination with a thyroid hormone, disclosed herein may include individually a variety of enteral or parenteral approaches including, without limitation, oral administration in any acceptable form, such as tablet, liquid, capsule, powder, or the like; topical administration in any acceptable form, such as drops, spray, creams, gels or ointments; buccal, nasal, and/or inhalation administration in any acceptable form; rectal administration in any acceptable form; vaginal administration in any acceptable form; intravascular administration in any acceptable form, such as intravenous bolus injection, intravenous infusion, intra-arterial bolus injection, intra-arterial infusion and catheter instillation into the vasculature; peri- and intra-tissue administration in any acceptable form, such as intraperitoneal injection, intramuscular injection, subcutaneous injection, subcutaneous infusion, intraocular injection, retinal injection, or sub-retinal injection or epidural injection; intravesicular administration in any acceptable form, such as catheter instillation; and by placement device, such as an implant, a stent, a patch, a pellet, a catheter, an osmotic pump, a suppository, a bioerodible delivery system, a non-bioerodible delivery system or another implanted extended or slow release system. An exemplary list of biodegradable polymers and methods of use are described in, e.g., Handbook of Biodegradable Polymers (Abraham J. Domb et al., eds., Overseas Publishers Association, 1997).

A compound, a composition, or a combination disclosed herein may be administered to a mammal using a variety of routes. Routes of administration suitable for treating a cancer as disclosed herein include both local and systemic administration. Local administration results in significantly more delivery of a compound, a composition, or a combination to a specific location as compared to the entire body of the mammal, whereas, systemic administration results in delivery of a compound, a composition, or a combination to essentially the entire body of the individual.

The actual route of administration of a compound, a composition, or a combination disclosed herein used can be determined by a person of ordinary skill in the art by taking into account factors, including, without limitation, the duration of treatment desired, the degree of relief desired, the duration of relief desired, the particular compound, composition, or combination, the rate of excretion of the compound, composition, or combination used, the pharmacodynamics of the compound, composition, or combination used, the nature of the other compounds to be included in the composition or combination, the particular route of administration, the particular characteristics, history and risk factors of the individual, such as, age, weight, general health and the like, the response of the individual to the treatment, or any combination thereof. An effective dosage amount of a compound, a composition, or a combination disclosed herein can thus readily be determined by the person of ordinary skill in the art considering all criteria and utilizing his best judgment on the individual's behalf.

In an embodiment, a compound, a composition, or a combination disclosed herein is administered systemically to a mammal. In another embodiment, a compound, a composition, or a combination disclosed herein is administered locally to a mammal. In an aspect of this embodiment, a compound, a composition, or a combination disclosed herein is administered to the site of the cancer of a mammal.

In other embodiments, RXR agonists may be administered orally, buccally, by nasal, and/or inhalation administration, intravascularly, intravenously, by intraperitoneal injection, intramuscularly, subcutaneously, intraocularly injection, by epidural injection, or by intravesicular administration; and thyroxine may be administered orally or subcutaneously or by another route. The RXR agonists, and the thyroid hormone do not need to be administered by the same route or on the same administration schedule.

Aspects of the present specification provide, in part, administering a therapeutically effective amount of a RXR agonist in combination with a thyroid hormone. As used herein, the term “therapeutically effective amount” is synonymous with “therapeutically effective dose” and when used in reference to treating a cancer means a dose of a compound, a composition, or a combination necessary to achieve the desired therapeutic effect and includes a dose sufficient to reduce tumor burden or place a patient into a clinical remission. The amount of active component in a compound, composition, or combination disclosed herein for treating a cancer may be varied so that a suitable dosage is obtained.

Additionally, where repeated administration of a compound, a composition, or a combination disclosed herein is used, the actual effect amount of compound, composition, or combination disclosed herein will further depend upon factors, including, without limitation, the frequency of administration, the half-life of the compound, composition, or combination disclosed herein. It is known by a person of ordinary skill in the art that an effective amount of a compound or a composition disclosed herein can be extrapolated from in vitro assays and in vivo administration studies using animal models prior to administration to humans. Wide variations in the necessary effective amount are to be expected in view of the differing efficiencies of the various routes of administration. For instance, oral administration generally would be expected to require higher dosage levels than administration by intravenous or intravitreal injection. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors.

As a non-limiting example, when administering a RXR agonists disclosed herein to a mammal, a therapeutically effective amount generally may be in the range of about 0.001 mg/day to about 3000 mg/day. In aspects of this embodiment, an effective amount of a compound or a composition disclosed herein may be about 0.01 mg/day to about 0.1 mg/day, about 0.03 mg/day to about 3.0 mg/day, about 0.1 mg/day to about 3.0 mg/day, about 0.3 mg/day to about 3.0 mg/day, about 1 mg/day to about 3 mg/day, about 3 mg/day to about 30 mg/day, about 10 mg/day to about 30 mg/day, about 10 mg/day to about 100 mg/day, about 30 mg/day to about 100 mg/day, about 100 mg/day to about 1000 mg/day, about 100 mg/day to about 300 mg/day, or about 1000 mg/day to about 3000 mg/day. In yet other aspects of this embodiment, a therapeutically effective amount of a compound or a composition disclosed herein may be at least 0.001 mg/kg/day, at least 0.01 mg/day, at least 0.1 mg/day, at least 1.0 mg/day, at least 3.0 mg/day, at least 10 mg/day, at least 30 mg/day, at least 100 mg/day, at least 300 mg/day, or at least 1000 mg/day. In yet other aspects of this embodiment, a therapeutically effective amount of a compound or a composition disclosed herein may be at most 0.001 mg/day, at most 0.01 mg/day, at most 0.1 mg/day, at most 1.0 mg/day, at most 3.0 mg/day, at most 10 mg/day, at most 30 mg/day, at most 100 mg/day, at most 300 mg/day, at most 1000 mg/day, or at most 3000 mg/day.

Suitable thyroxine doses are generally from about 12.5 μg/day to about 250 μg/day orally initially with an increase in dose of about 12.5 to about 25 μg daily increments every 2-4 weeks as needed. In other embodiments, the suitable thyroxine dose is from about 5 μg/day to about 225 μg/day, from about 7.5 μg/day to about 200 μg/day, from about 10 μg/day to about 175 μg/day, from about 12.5 μg/day to about 150 μg/day, from about 15 μg/day to about 125 μg/day, from about 17.5 μg/day to about 100 μg/day, from about 20 μg/day to about 100 μg/day, from about 22.5 μg/day to about 100 μg/day, from about 25 μg/day to about 100 μg/day, from about 5 μg/day to about 200 μg/day, from about 5 μg/day to about 100 μg/day, from about 7.5 μg/day to about 90 μg/day, from about 10 μg/day to about 80 μg/day, from about 12.5 μg/day to about 60 μg/day, or from about 15 μg/day to about 50 μg/day. Increases in dose are generally made in increments of about 5 μg/day, about 7.5 μg/day, about 10 μg/day, about 12.5 μg/day, about 15 μg/day, about 20 μg/day, or about 25 μg/day. In certain embodiments, the suitable thyroid hormone dose is a dose able to produce serum levels of T4 in the top 50%, the top 60%, the top 70%, the top 80%, or the top 90% of the normal range for the testing laboratory. As the normal range of T4 levels may vary by testing laboratory, the target T4 levels are based on normal ranges determined for each particular testing laboratory.

Dosing may be single dosage or cumulative (serial dosing), and may be readily determined by one skilled in the art. For instance, treatment of a cancer may comprise a one-time administration of an effective dose of a compound, composition, or combination disclosed herein. As a non-limiting example, an effective dose of a compound, composition, or combination disclosed herein can be administered once to a mammal as a single injection or deposition at or near the site exhibiting a symptom of a cancer or a single oral administration of the compound, composition, or combination. Alternatively, treatment of a cancer may comprise multiple administrations of an effective dose of a compound, composition, or combination disclosed herein carried out over a range of time periods, such as daily, once every few days, weekly, monthly or yearly. As a non-limiting example, a compound, a composition, or a combination disclosed herein may be administered once or twice weekly to a mammal. The timing of administration can vary from mammal to mammal, depending upon such factors as the severity of a mammal's symptoms. For example, an effective dose of a compound, composition, or combination disclosed herein can be administered to a mammal once a month for an indefinite period of time, or until the mammal no longer requires therapy. A person of ordinary skill in the art will recognize that the condition of the mammal can be monitored throughout the course of treatment and that the effective amount of a compound, composition, or combination disclosed herein that is administered can be adjusted accordingly.

In other embodiments, the method may further include measuring the patient's Cₘₐₓ of the RXR agonist and adjusting the dose to maintain the patient's Cₘₐₓ at an optimal level.

In some embodiments, the method further includes treating the patient with one or more triglyceride lowering agents.

Examples of cancers which can be treated by the disclosed methods may include, but are not limited to, acute lymphoblastic leukemia, acute myeloid leukemia, adrenocortical carcinoma, AIDS-related lymphoma, an AIDS-related malignancy, anal cancer, bile duct cancer, bladder cancer, bone cancer, brain stem glioma, a brain tumor (e.g., astrocytoma, cerebellar astrocytoma; cerebral astrocytoma/malignant glioma, pendymoma brain tumor, supratentorial primitive brain tumor, a neuroectodermal tumor, visual pathway and hypothalamic glioma, etc.), breast cancer, a bronchial adenoma/carcinoid, carcinoid tumor, carcinoma (adrenocortical, gastrointestinal, islet cell, skin, unknown primary, etc.); cervical cancer, a childhood cancer, chronic lymphocytic leukemia, chronic myelogenous leukemia, a chronic myeloproliferative disorder, clear cell sarcoma of tendon sheaths, colon cancer, colorectal cancer, cutaneous T-cell lymphoma, endometrial cancer, ependymoma, epithelial cancer, esophageal cancer, a Ewing family of tumor, extracranial germ cell tumor, extragonadal germ cell tumor, extrahepatic bile duct cancer, eye cancer, intraocular melanoma, gallbladder cancer, gastric cancer, gastrointestinal carcinoid tumor, ovarian germ cell tumor, gestational trophoblastic tumor, hairy cell leukemia, head and neck cancer, hepatocellular cancer, Hodgkin's lymphoma, hypopharyngeal cancer, islet cell carcinoma (endocrine pancreas), Kaposi's sarcoma, kidney cancer, laryngeal cancer, lip and oral cavity cancer, primary liver cancer, lung cancer, non-small cell lung cancer, small cell lung cancer, primary central nervous system lymphoma, Non-Hodgkin's lymphoma, Waldenstrom's macroglobulinemia, malignant mesothelioma, malignant thymoma, medulloblastoma, melanoma, Merkel cell carcinoma, primary metastatic squamous neck cancer, multiple endocrine neoplasia syndrome, multiple myeloma/plasma cell neoplasm, mycosis fungoides, a myelodysplastic syndrome, multiple myeloma, nasal cavity and paranasal sinus cancer, nasopharyngeal cancer, neuroblastoma, oral cancer, oropharyngeal cancer, ovarian epithelial cancer, ovarian low malignant potential tumor, pancreatic cancer, parathyroid cancer, pheochromocytoma penile cancer, pineal and supratentorial primitive neuroectodermal tumors, pituitary tumor, pleuropulmonary blastoma; prostate cancer, rectal cancer, renal cancer, transitional cell cancer, retinoblastoma, rhabdomyosarcoma, salivary gland cancer, sarcoma (e.g., Ewing's family of tumors, Kaposi's sarcoma, osteosarcoma/malignant fibrous histiocytoma of bone, soft tissue, etc.), Sezary syndrome, skin cancer, small intestine cancer, testicular cancer, thymoma, thyroid cancer, trophoblastic tumor, vaginal cancer, vulvar cancer, or Wilms' Tumor. In other embodiments, the method may treat lung cancers, prostate cancer, breast cancer, and/or pancreatic cancer.

There are two standard methods for the evaluation of oncology treatment response: the WHO and RECIST standards. These methods measure a tumor to compare a current tumor with past measurements or to compare changes with future measurements and make to make changes in a treatment regimen. In the WHO method, the tumor's long and short axes are measured and the product of these two measurements is then calculated; if there are multiple tumors, the sum of all the products is calculated. In the RECIST method, only the long axis is measured. If there are multiple tumors, the sum of all the long axes measurements is calculated. However, with lymph-nodes, the short axis is measured instead of the long axis.

In some embodiments of the current method, the tumor size of a patient treated with a compound or combination disclosed herein is reduced by about 5%, about 10%, about 15%, about 20%, about 25%, about 30%, about 35%, about 40%, about 45%, about 50%, about 55% about 60%, about 65%, about 70%, about 75%, about 80%, about 90%, about 95%, about 100%, or any other range bound by these values.

In other embodiments, the 1-year survival rate of an individual treated with a compound or combination disclosed herein is increased by about 5%, about 10%, about 15%, about 20%, about 25%, about 30%, about 35%, about 40%, about 45%, about 50%, about 55% about 60%, about 65%, about 70%, about 75%, about 80%, about 90%, about 95%, about 100%, or any other range bound by these values.

In other embodiments, the 5-year survival rate of an individual treated with a compound or combination disclosed herein is increased by about 5%, about 10%, about 15%, about 20%, about 25%, about 30%, about 35%, about 40%, about 45%, about 50%, about 55% about 60%, about 65%, about 70%, about 75%, about 80%, about 90%, about 95%, about 100%, or any other range bound by these values.

In other embodiments, the 10-year survival rate of an individual treated with a compound or combination disclosed herein is increased by about 5%, about 10%, about 15%, about 20%, about 25%, about 30%, about 35%, about 40%, about 45%, about 50%, about 55% about 60%, about 65%, about 70%, about 75%, about 80%, about 90%, about 95%, about 100%, or any other range bound by these values

In other embodiments, the method may help to treat or alleviate conditions, symptoms, or disorders related to cancer. In some embodiments, these conditions or symptoms may include, but are not limited to, anemia, asthenia, cachexia, Cushing's Syndrome, fatigue, gout, gum disease, hematuria, hypercalcemia, hypothyroidism, internal bleeding, hair loss, mesothelioma, nausea, night sweats, neutropenia, paraneoplastic syndromes, pleuritis, polymyalgia rheumatica, rhabdomyolysis, stress, swollen lymph nodes, thrombocytopenia, Vitamin D deficiency, or weight loss. In other embodiments, the administration of the combination of the RXR agonist with the thyroid hormone prolongs the survival of the individual being treated.

A compound, composition, or combination disclosed herein as disclosed herein can also be administered to a mammal in combination with other therapeutic compounds to increase the overall therapeutic effect of the treatment. The use of multiple compounds to treat an indication can increase the beneficial effects while reducing the presence of side effects.

Aspects of the present specification may also be described as follows:

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the methods of treating a cancer using a RXR agonist disclosed herein, in combination with a thyroid hormone, uses of a RXR agonist disclosed herein and a thyroid hormone to manufacture a medicament to treat a cancer.

### Example 1

### Selective RXR Agonist, IRX4204, Exerts its Biological Effects through RXR Signaling

To determine whether a RXR agonist can mediate its effects via RXRα receptor homodimers, RXRβ receptor homodimers, RXRγ receptor homodimers, or any combination thereof, or the corresponding RAR/RXR heterodimers, receptor-mediated transactivation assays were performed. For transactivation assays assessing RXR homodimer signaling, CV-1 cells were transfected with 1) an expression construct including a full length RXRα, RXRβ, or RXRγ; and 2) a rCRBPII/RXRE-tk-Luc reporter construct that included RXR homodimer-specific RXRE/DR1 responsive element linked to a luciferase gene. For transactivation assays assessing RAR/RXR heterodimer signaling, CV-1 cells were transfected with 1) an expression construct comprising a fusion protein including an estrogen receptor (ER) DNA binding domain linked to the ligand binding domain of RARα, RARβ, or RARγ and 2) a ERE-tk-Luc reporter construct that included an estrogen receptor responsive element linked to a luciferase gene. The ER-RAR fusion proteins provided an accurate readout of only the transfected ER-RAR. After transfection, CV-1 cells were treated with RXR agonist IRX4204 at increasing concentrations for 20 hours before measuring luciferase activity. Luciferase activity is expressed as percent of maximal activity obtained using 1 μM RXR agonist IRX4204 for RXRs and 1 μM all-trans-retinoic acid (ATRA) for RARs (Table 1). Data are mean values±SE from five independent experiments.

TABLE 1
RXR Agonist Potencies in Activating RXRs and RARs

| | | EC₅₀ (nM) | | | EC₅₀ (nM) | | |
|---|---|---|---|---|---|---|---|
| | | Efficacy (% of 1 μM IRX4204) | | | Efficacy (% of 1 μM ATRA) | | |
| Compound | Structure | RXRα | RXRβ | RXRγ | RARα | RARβ | RARγ |
| IRX4204 | 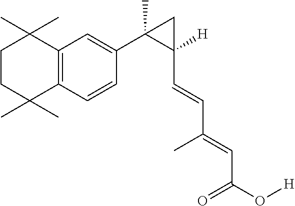 [SMILES: CC(/C=C/[C@@H]1C[C@]1(C)c1ccc2c(c1)C(C)(C)CCC2(C)C)=C\C(=O)O] | 0.08 ± 0.01 100 | 0.47 ± 0.05 100 | 0.09 ± 0.01 100 | >1,000 | >1,000 | >1,000 |

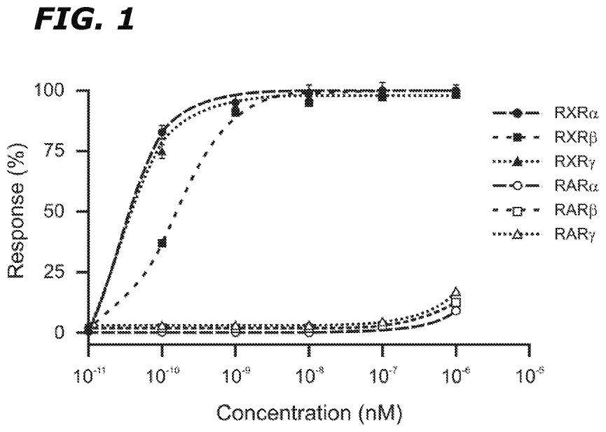

These results indicate that RXR agonist IRX4204 activated RXR receptors with very high potency (EC₅₀<0.5 nM) for all three RXR subtypes (Table 1). In contrast, EC₅₀ of the RXR agonist for RARs was >1,000 nM with minimal activity detected at ≥1 μM. This difference represents >2,000-fold selectivity for RXRs over RARs in functional transactivation assays. Additionally, these data demonstrate that RXR agonist IRX4204 was more than 1,000-fold more potent in activating RXR receptors rather than RAR receptors. These results indicate that the biological effects of selective agonists such as IRX4204 are mediated through a RXR signaling pathway and not via a RAR signaling pathway. Also, using appropriate receptor and reporter constructs, RXR agonist IRX4204 was shown not to transactivate so called “permissive RXR heterodimers” PPAR/RXR, FXR/RXR and LXR/RXR (FIGS. 1A-C). In this regard, RXR agonist IRX4204 is distinct from other RXR agonists. Additionally, IRX4204 selectively activates the Nurr1/RXR permissive heterodimer (FIG. 1D). Thus, RXR agonist IRX4204 has a unique profile in that it selectively activates only RXR homodimers and Nurr1/RXR heterodimers.

### Example 2

### Binding Affinity of RXR Agonists

To determine the binding affinity for a RXR agonist, competitive displacement assays were performed. RXRα, RXRβ, RXRγ, RARα, RARβ, or RARγ were expressed in SF21 cells using a baculovirus expression system and the resulting proteins were purified. To determine the binding affinity for a RXR agonist for an RXR, purified RXRα, RXRβ, and RXRγ were separately incubated with 10 nM [³H]-9CRA, and the binding affinity of the RXR agonist IRX4204 was determined by competitive displacement of [³H]-9CRA from the receptor. To determine the binding affinity for a RXR agonist for an RAR, purified RARα, RARβ, and RARγ were incubated with 5 nM [³H]-ATRA, and the binding affinity of the RXR agonist IRX4204 was determined by competitive displacement of [³H]-ATRA from the receptor. Ki values are mean values of at least two independent experiments (Table 2). Standard errors (±) among independent experiments are indicated.

As shown in Table 2, RXR agonist IRX4204 displayed high affinity for RXRα, RXRβ, and RXRγ with Ki values being 1.7, 16, and 43 nM, respectively. In contrast, the RXR agonist IRX4204 bound with very low affinity to each of the RARs (Ki values being >1,000 nM). These data indicate that IRX4204 is highly selective for the RXRs relative to the RARs.

TABLE 2
RXR Agonist Binding Affinities

| | | RXR Binding Affinity | | | RAR Binding Affinity | | |
|---|---|---|---|---|---|---|---|
| | | Ki (nM) | | | Ki (nM) | | |
| Compound | Structure | RXRα | RXRβ | RXRγ | RARα | RARβ | RARγ |
| IRX4204 | 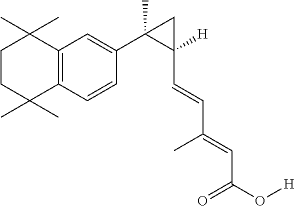 [SMILES: CC(/C=C/[C@@H]1C[C@]1(C)c1ccc2c(c1)C(C)(C)CCC2(C)C)=C\C(=O)O] | 1.7 ± 0.1 | 16 ± 1.0 | 43 ± 3.0 | 6344 ± 674 | 7552 ± 638 | 4742 ± 405 |

### Example 3

### RXR Agonist IRX4204 as a Selective Activator of Nurr1/RXR Permissive Heterodimer

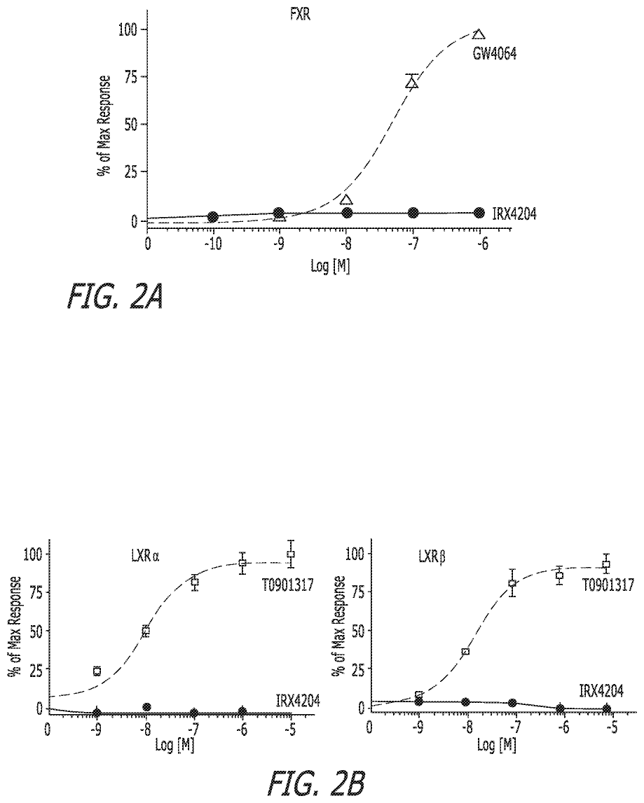

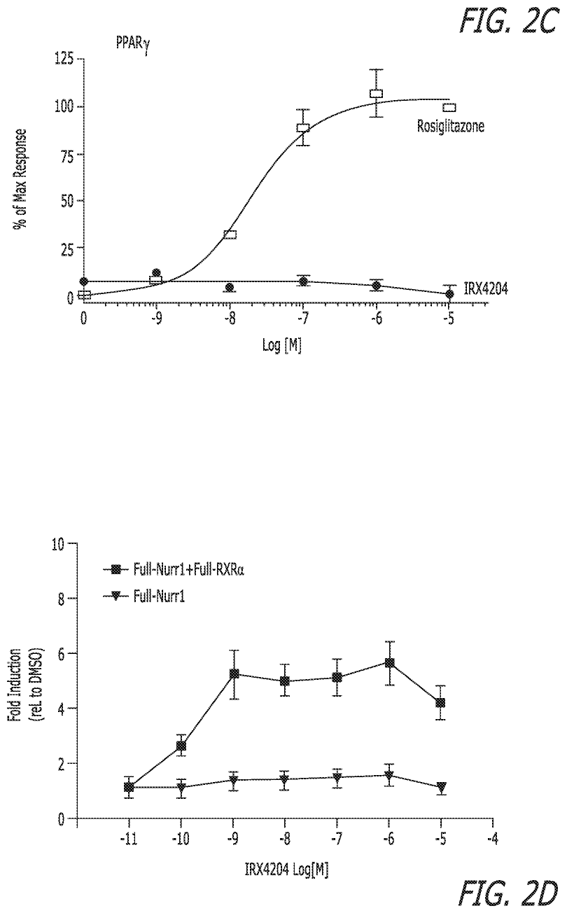

In order to determine which permissive RXR heterodimer is activated by the RXR agonist IRX4204, receptor transactivation assays were carried out as follows for PPARγ/RXR, FXR/RXR, LXRα/RXR, LXRβ/RXR, and Nurr1/RXR. For PPARγ: CV-1 cells were transfected with 3×(rAOX/DR1)-tk-Luc reporter gene and an expression vector for PPARγ. For FXR:CV-1 cells were transfected with 3×(IBABP/IRI)-tk-Luc reporter gene and vectors for FXR and RXRα. For LXR:CV-1 cells were transfected with 3×(PLTP/LXRE)-tk-Luc reporter gene with vectors for LXRα or LXRβ. For Nurr1: COS7 cells were transfected with 3×NBRE-tk-luc reporter gene and full length Nurr-1 with or without full-length RXRα plasmid. Cells were then treated with vehicle or IRX4204 for 20 hr. Luciferase data were normalized to co-transfected β-gal activity. Luciferase activity was expressed as percent of maximal activity obtained using specific agonists. Rosiglitazone (PPARγ), GW4064 (FXR), T0901317 (LXR). The data indicate that IRX4204 does not activate FXR/RXR (FIG. 2A), LXRα/RXR or LXRβ/RXR (FIG. 2B), or PPARγ/RXR (FIG. 2C). In contrast, IRX4204 potently (EC₅₀<1 nm) activates the Nurr1/RXR heterodimer (FIG. 2D). These data collectively indicate that IRX4204 is a unique RXR agonist in that it selectively activates the Nurr1/RXR heterodimer but not the PPARγ/RXR, FXR/RXR or LXR/RXR heterodimers.

### Example 4

### In Vitro Growth Inhibition in Cancer Cells

The inhibition of growth was tested in a variety of cancer cell lines. The methods utilized are described below and the experimental results are summarized in Table 3 below.

All cell lines were obtained from American Type Culture Collection (ATCC, Manassas, Va.) and culture media and supplements were supplied by ATCC and Invitrogen (Carlsbad, Calif.). The ATP assay was performed using CELLTITER-GLO Luminescent Cell Viability Assay Kit from Promega (Madison, Wis.) which allows ATP to be roughly quantified based on the fluorescence emitted by the cells. Luminescence was recorded using VICTOR-22 1420 (Perkin Elmer, Waltham, Mass.).

The thymidine incorporation assay allows for specific detection of [³H]-labeled thymidine (Perkin-Elmer) incorporated into newly synthesized DNA. Labeled cells were collected and their DNA was harvested on to a fiberglass filter using MLR24 Cell Harvester (Brandel, Geithersburg, Md.). The amount of incorporated [³H] thymidine was determined by scintillation counting using Trilux-1450 Microbeta Counter (Perkin-Elmer).

The soft-agar coupled thymidine incorporation assay is used to determine the ability of cells to form colonies through invasion into agar matrix. A bottom feeder layer of cells was composed of 0.6% NUSIEVE GTG agarose (Cambrex, East Rutherford, N.J.), D-MEM with low glucose, 2 mM L-glutamine, and 15% charcoal/dextran-treated FBS. Test cells were seeded in the top soft-agar layer containing 0.3% NUSIEVE GTG agarose, D-MEM (low glucose), 2 mM L-glutamine, and 15% charcoal/dextran-treated FBS at a density of 10,000-20,000 cells per well. Test cells were labeled with [³H] thymidine at 50 nM. Labeled cells were collected and counted as described above.

For the soft-agar colony formation assay, cells were cultured in 6-well plates containing two agar layers with growth medium. The bottom feeder layer contained 0.5% DIFCO Noble Agar (Becton Dickinson & Co., East Rutherford, N.J.), D-MEM with low glucose and 2 mM L-glutamine, and 15% charcoal/dextran-treated FBS. Test cells were seeded in the top soft-agar layer containing 0.3% agarose, D-MEM (low glucose), 2 mM L-glutamine, and 15% charcoal/dextran-treated FBS at a density of 10,000 cells per well. After treatment with drugs, cell colonies were counted under a light microscope.

IC₅₀ represents the concentration required to obtain 50% of the maximal cell growth inhibition by IRX4204. Efficacy is the maximal inhibition of cell growth at the maximal concentration used in the studies. ND means the results were not determined. The experimental results show IRX4204 as a potent compound in inhibiting cancer cell growth.

TABLE 3

| | Cell | | | | Efficacy | |
|---|---|---|---|---|---|---|
| Cancer Type | Line | Method | IC₅₀ | | (Conc.) | |
| Cutaneous | HuT 78 | ATP | 1.6 | nM | 81% | (1 μM) |
| T-Cell | | | | | | |
| Lymphoma | | | | | | |
| Small Cell | H-82 | [H3] | 3.6 | nM | 72% | (1 μM) |
| Lung Cancer | | Thymidine | | | | |
| | | Incorporation | | | | |
| | H446 | Soft-Agar | 17 | nM | 44% | (1 μM) |
| Non-Small Cell | A-427 | ATP | 7.7 | nM | 50% | (10 μM) |
| Lung Cancer | | | | | | |
| Breast, ER- | T47D | ATP | 75 | nM | 97% | (1 μM) |
| positive | | Soft-Agar | 12.6 | nM | 97% | (10 μM) |
| | MCF-7 | ATP | 52 | nM | 52% | (1 μM) |
| Breast, ER- | SK-BR-3 | ATP | 3 | nM | 74% | (1 μM) |
| negative | | | | | | |
| Leukemia | HL-60 | [H³] | 15 | nM | 94% | (1 μM) |
| | | Thymidine | | | | |
| | | Incorporation | | | | |
| | U937 | ATP | 0.73 | nM | 94% | (1 μM) |
| Cervical | HT-3 | ATP | <1 | μM | 68% | (1 μM) |
| | SiHa | ATP | 47 | nM | 41% | (1 μM) |
| | ME-180 | ATP | 7 | nM | 80% | (1 μM) |
| Colon | SW403 | ATP | 0.78 | nM | 35% | (1 μM) |

| | | | | |
|---|---|---|---|---|
| SW48 | Cell Counting | ND | 60% | (1 μM) |

### Example 5

### Effects RXR Agonists on NSCLC Tumors

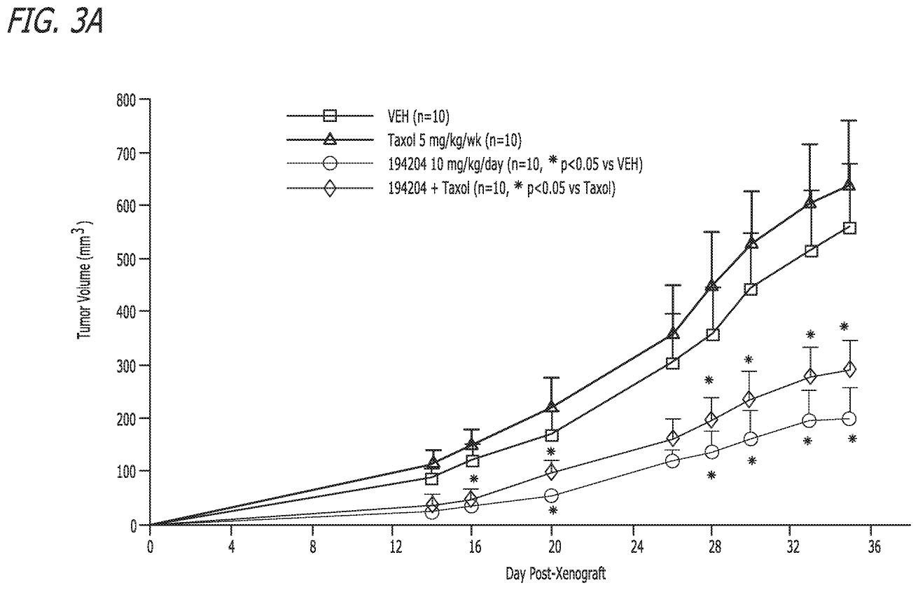

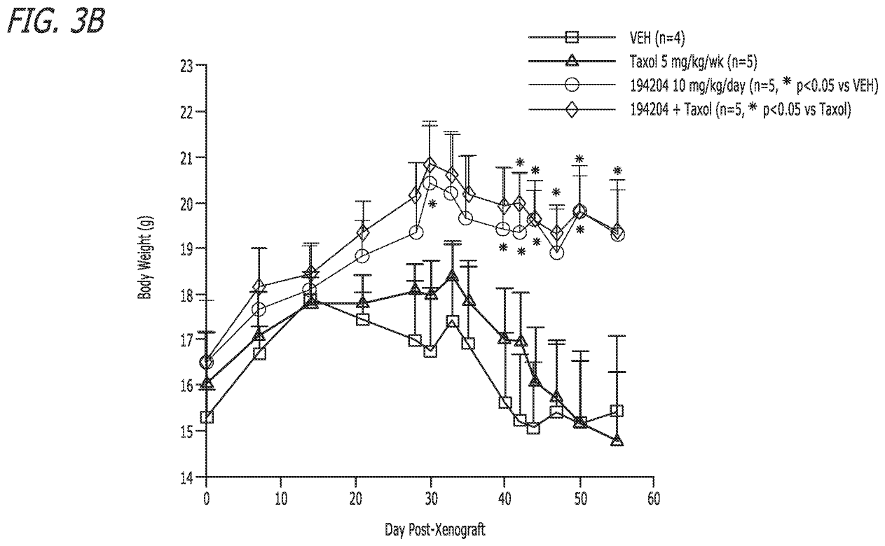

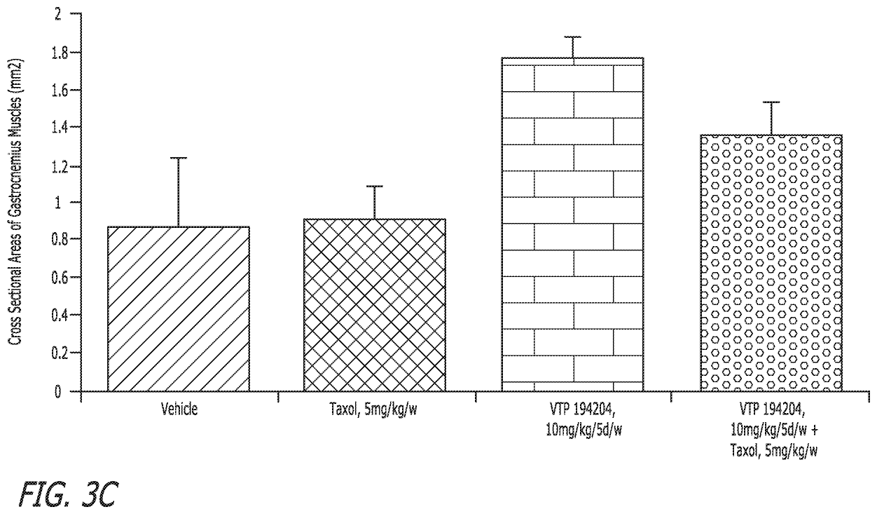

The study showed the effects of IRX4204 on nude mice xenografted with human H292 NSCLC tumors. Nude mice were randomized into 4 groups of 10 animals each based on body weight and xenografted subcutaneously in the right flank with H292 cells (2×10⁶ cells). Drug treatment was started immediately after xenografting and continued for 35 days (5 animals of each group) or 55 days (remaining 5 animals). The animals were treated with vehicle (VEH); TAXOL 5 mg/kg/week, once a week, i.p.; IRX4204 10 mg/kg/day, 5 days a week, by oral gavage; or IRX4204+TAXOL. Tumor sizes were measured periodically for 35 days (FIG. 3A). Animals #1-5 of each group were sacrificed after 35 days of treatment and gastrocnemus muscles were determined (FIG. 3C). The body weights and overall appearance of animals #6 through #10 from each group were followed for an extended period (FIG. 3B).

### Example 6

### Phase I/II Study of IRX4204 in Refractory Malignancies in Humans

The study investigated once-daily oral administration of IRX4204 in patients with refractory malignancies. Test groups were dosed with 3, 6, 12, and 24 mg/m² IRX4204 for a minimum of four weeks; a longer treatment schedule was available to patients showing disease stabilization. Data was collected for 18 patients with progressive, solid tumors. During the course of the investigation, there were no serious adverse events considered possibly or probably related to IRX4204. Adverse events which were recorded included triglyceride elevation and hypothyroidism which are known biologic effects of RXR agonism.

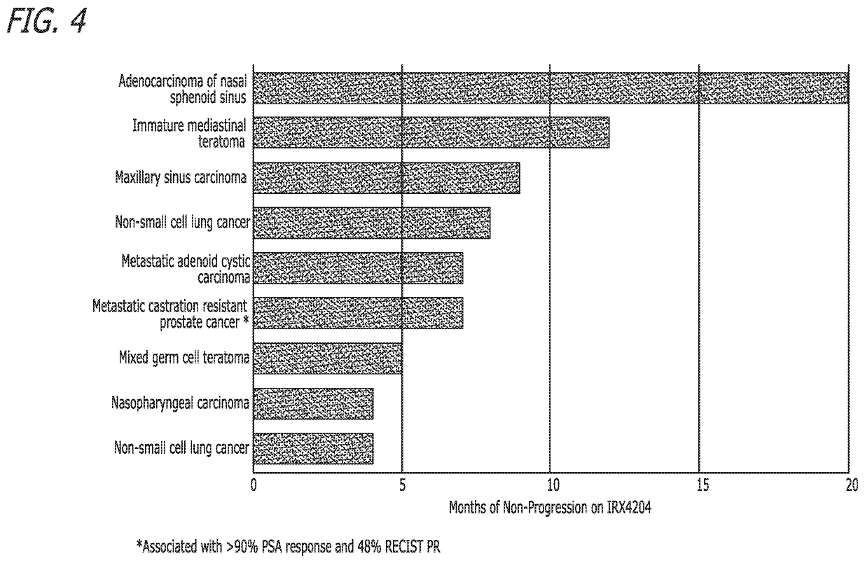

FIG. 4 shows 50% of the patients (i.e., 9 patients) did not exhibit tumor progression for at least four months when treated with IRX4204.

### Example 7

### RXR Activation Threshold

While not intending to be bound by any particular theory, one aspect of the instant disclosure contemplates that bexarotene particularly at high doses, activates RARs in addition to RXRs and this activation of RAR is why the non-responsive group (those with low triglyceride elevation) had decreased survival in the pivotal bexarotene clinical trials (the SPIRIT trials).

At a dose of 400 mg/m²/d, bexarotene has Cₘₐₓ values that are estimated to be around 8,000 nM in the blood, at which concentration there will be significant activation of RARs by bexarotene. Also, since the estimated bexarotene Cₘₐₓ values are around 2,000 nM at the lower dose (225 mg/m²/d), some detrimental activation of RARs would occur even at this dose.

Accordingly, it is envisioned that a patient could be dosed with bexarotene to determine the RAR activating threshold and the RXR effective dose for the patient; administering to the patient bexarotene at a level below the RAR activating threshold and at or above the RXR effective dose.

### Example 8

### Efficacy of IRX4204 With and Without Thyroid Hormone Replacement in a Xenograft

BALB/c mice that are approximately 8-12 weeks old are used in this study. Animals are randomized into treatment groups based on body weight. The weight of the mice is measured at the start at the beginning of the study and subsequently biweekly until the end of the study. The mice are injected with 1×10⁷ H292 tumor cells in MATRIGEL subcutaneously in the flank with a cell injection volume of 0.1 mL/mouse. A pair match is performed when the tumors reach an average size of 100-150 mm³ and treatment with test agents is started. The test mice are dosed according to the regimen as described in Table 4 below.

TABLE 4

| | | Regimen 1 | | | | Regimen 2 | | | |
|---|---|---|---|---|---|---|---|---|---|
| Gr. | N | Agent | μg/kg | Route | Schedule | Agent | μg/kg | Route | Schedule |
| 1^{#} | 10 | vehicle 1 | — | sc | qd × 7 | vehicle 2 | — | po | qd × 7 |
| 2 | 10 | vehicle 1 | — | sc | qd × 7 | IRX4204 | 10 | po | qd × 7 |
| 3 | 10 | Thyroxine | 20 | sc | qd × 7 | IRX4204 | 10 | po | qd × 7 |
| 4 | 10 | Thyroxine | 20 | sc | qd × 7 | vehicle 2 | — | po | qd × 7 |

#- Control Group

During the study, caliper measurements of the tumor size are taken biweekly until the end of the study. Any individual animal with a single observation of >than 30% body weight loss or three consecutive measurements of greater than 25% body weight loss is removed from the study. Any group with a mean body weight loss of greater than 20% or a mortality of greater than 10% is stopped being dosed and may be allowed to recover. Within a group with more than 20% weight loss, individuals hitting the individual body weight loss endpoint are removed from the study. If the group treatment related body weight loss is recovered to within 10% of the original weights, dosing may resume at a lower dose or less frequent dosing schedule. Exceptions to non-treatment body weight percentage recovery are allowed on a case-by-case basis.

Animals are monitored individually. The endpoint of the experiment is a tumor volume of 1500 mm³ or 45 days, whichever comes first. Animals that respond to the IRX4204 containing treatments can be followed longer.

Blood samples will be collected by terminal cardiac puncture under isoflurane anesthesia. Blood samples are separated into components with the blood plasma dosed with an anti-coagulant and K2EDTA, and preserved at −4° C. The plasma are then sent for T4 assays.

### Example 9

### A Randomized Phase III Clinical Trial Comparing IRX4204/Carboplatin/Paclitaxel/Thyroxine versus IRX4204/Carboplatin/Paclitaxel in Chemotherapy—Naïve Patients with Advanced or Metastatic Non-Small Cell Lung Cancer (NSCLC)

IRX4204, a second generation rexinoid, is a potent, specific agonist at the retinoid X receptors (RXRs) Because of its very high selectivity for the RXRs relative to the RARs, IRX4204 can readily be used in the clinic at doses which activate the RXRs and not the RARs. IRX 4204 exhibited tumor growth inhibitory effects in cell lines derived from a variety of tumor types and in animal models of breast, lung, and prostate cancer. Phase III clinical trials demonstrate the survival benefit of the addiction of IRX4204 to carboplatin/paclitaxel chemotherapy. This study is designed to determine a possible clinical benefit of the addition of thyroxine to the IRX4204/ carboplatin/paclitaxel combination in chemotherapy-naïve Stage IIIB or Stage IV NSCLC patients.

Study Design: Patient Selection. Patients are selected to meet all following conditions: (a) NSCLC at Stage IIIB with pleural effusion or Stage IV; (b) No prior chemotherapy; and (c) ECOG=0, 1.

Patients stratified by disease stage and gender are randomized to receive once daily thyroxine in combination with IRX4204 at a pure RXR-activating dose and carboplatin AUC 6 and paclitaxel 200 mg/m² (every 3 weeks) or IRX4204/carboplatin/paclitaxel alone.

Study Endpoints: The primary endpoint is overall survival and the secondary endpoint is Kaplan-Meier projected two-year survival rate.

The predicted, not actual, clinical results are presented in Table 5:

TABLE 5

| | Median | Two-Year |
|---|---|---|
| Treatment Group | Survival (Months) | Survival Rates (%) |
| IRX4204 in combination with | 15 | 30 |
| Carboplatin/Paclitaxel | | |
| IRX4204 in combination with | 38 | 62 |
| thyroxine/carboplatin/paclitaxel | | |

A Phase II/III registration study in fourth-line NSCLC patients (n=~160) will compare IRX4204 in combination with a thyroid hormone versus best supportive care with crossover upon disease progression. Approval will be based on a primary endpoint of progression-free survival. Subsequent or parallel studies will seek to expand clinical indications for IRX4204 and thyroid hormone in NSCLC with combinations of platinum-based therapies or TARCEVA and in other cancers.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method of treating a cancer, the method comprising administering to an individual in need thereof a therapeutically effective amount of a RXR agonist and a thyroid hormone, wherein the RXR agonist has the structure of Formula II 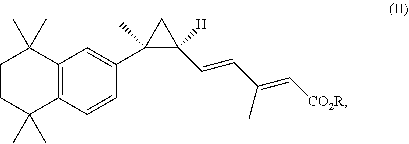
wherein R is H or lower alkyl of 1 to 6 carbons, or an ester a pharmaceutically acceptable salt thereof, bexarotene, or LG268;
wherein administration of the combination of the RXR agonist and thyroid hormone treats the cancer in the individual more effectively than the RXR agonist alone.

2. The method according to claim 1, wherein the RXR agonist is a selective RXR agonist comprising 3,7-dimethyl-6(S),7(S)-methano,7-[1,1,4,4-tetramethyl-1,2,3,4-tetrahydronaphth-7-yl]2(E),4(E) heptadienoic acid.

3. The method according to claim 1, wherein the RXR agonist is a selective RXR agonist comprising 3,7-dimethyl-6(S),7(S)-methano,7-[1,1,4,4-tetramethyl-1,2,3,4-tetrahydronaphth-7-yl]2(E),4(E) heptadienoic ethyl ester.

4. The method according to claim 1, wherein the RXR agonist is bexarotene.

5. The method according to claim 1, wherein the RXR agonist is LG268.

6. The method according to claim 1, wherein the thyroid hormone is thyroxine.

7. The method according to claim 1, wherein the therapeutically effective amount of the ester of the RXR agonist is about 0.001 mg/day to about 1000 mg/day.

8. The method according to claim 1, wherein the therapeutically effective amount of the RXR agonist is about 0.001 mg/day to about 1000 mg/day.

9. The method according to claim 1, wherein the therapeutically effective amount of the RXR agonist is about 10 mg/day to about 1000 mg/day.

10. The method according to claim 1, wherein the therapeutically effective amount of the RXR agonist is about 1 mg/day to about 50 mg/day.

11. The method according to claim 1, wherein the thyroid hormone is thyroxine and the dose of thyroxine is about 12.5 μg/day to about 250 μg/day.

12. The method according to claim 1, wherein the thyroid hormone is thyroxine and the dose of the thyroxine is a dose sufficient to maintain the individual's serum T4 levels at 50% to 80% of the normal range.

13. The method according to claim 1, wherein the RXR agonist is administered by nasal administration.

14. The method according to claim 13, wherein the RXR agonist and thyroxine are both administered by nasal administration.

15. The method according to claim 1, wherein the RXR agonist is administered orally.

16. The method according to claim 6, wherein the RXR agonist and the thyroxine are both administered substantially simultaneously.

17. The method according to claim 6, wherein the RXR agonist and the thyroxine are administered on different schedules.

18. The method according to claim 6, wherein the thyroxine is administered orally.

19. The method according to claim 6, wherein the thyroxine is administered subcutaneously

20. The method according to claim 1, wherein the cancer is acute lymphoblastic leukemia, acute myeloid leukemia, adrenocortical carcinoma, AIDS-related lymphoma, an AIDS-related malignancy, anal cancer, bile duct cancer, bladder cancer, bone cancer, brain stem glioma, a brain tumor (e.g., astrocytoma, cerebellar astrocytoma; cerebral astrocytoma/malignant glioma, pendymoma brain tumor, supratentorial primitive brain tumor, a neuroectodermal tumor, visual pathway and hypothalamic glioma, etc.), breast cancer, a bronchial adenoma/carcinoid, carcinoid tumor, carcinoma (adrenocortical, gastrointestinal, islet cell, skin, unknown primary, etc.); cervical cancer, a childhood cancer, chronic lymphocytic leukemia, chronic myelogenous leukemia, a chronic myeloproliferative disorder, clear cell sarcoma of tendon sheaths, colon cancer, colorectal cancer, cutaneous T-cell lymphoma, endometrial cancer, ependymoma, epithelial cancer, esophageal cancer, a Ewing family of tumor, extracranial germ cell tumor, extragonadal germ cell tumor, extrahepatic bile duct cancer, eye cancer, intraocular melanoma, gallbladder cancer, gastric cancer, gastrointestinal carcinoid tumor, ovarian germ cell tumor, gestational trophoblastic tumor, hairy cell leukemia, head and neck cancer, hepatocellular cancer, Hodgkin's lymphoma, hypopharyngeal cancer, islet cell carcinoma (endocrine pancreas), Kaposi's sarcoma, kidney cancer, laryngeal cancer, lip and oral cavity cancer, primary liver cancer, lung cancer, non-small cell lung cancer, small cell lung cancer, primary central nervous system lymphoma, Non-Hodgkin's lymphoma, Waldenstrom's macroglobulinemia, malignant mesothelioma, malignant thymoma, medulloblastoma, melanoma, Merkel cell carcinoma, primary metastatic squamous neck cancer, multiple endocrine neoplasia syndrome, multiple myeloma/plasma cell neoplasm, mycosis fungoides, a myelodysplastic syndrome, multiple myeloma, nasal cavity and paranasal sinus cancer, nasopharyngeal cancer, neuroblastoma, oral cancer, oropharyngeal cancer, ovarian epithelial cancer, ovarian low malignant potential tumor, pancreatic cancer, parathyroid cancer, pheochromocytoma penile cancer, pineal and supratentorial primitive neuroectodermal tumors, pituitary tumor, pleuropulmonary blastoma; prostate cancer, rectal cancer, renal cancer, transitional cell cancer, retinoblastoma; rhabdomyosarcoma, salivary gland cancer, sarcoma (e.g., Ewing's family of tumors, Kaposi's sarcoma, osteosarcoma/malignant fibrous histiocytoma of bone, soft tissue, etc.), Sezary syndrome, skin cancer, small intestine cancer, testicular cancer, thymoma, thyroid cancer, trophoblastic tumor, vaginal cancer, vulvar cancer, or Wilms' Tumor.

21. The method of claim 20, wherein the cancer is lung cancer.

22. The method of claim 20, wherein the cancer is prostate cancer.

23. The method of claim 20, wherein the cancer is breast cancer.

20. The method of claim 20, wherein the cancer is pancreatic cancer.

25. (canceled)

26. (canceled)

27. (canceled)

28. (canceled)

29. The method according to claim 1, further comprising administration of another anti-cancer agent.

29. The method according to claim 29, wherein the anti-cancer agent is TARCEVA (erlotinib), TAXOL (paclitaxel), TAXOTERE (docetaxel), dactinomycin, bleomycin, vinblastine, cisplatin, acivicin, aclarubicin, acodazole, acronine, adozelesin, aldesleukin, altretamine, ambomycin, ametantrone acetate; aminoglutethimide, amsacrine, anastrozole; anthramycin, asparaginase, asperlin, azacitidine, azetepa, azotomycin, batimastat, benzodepa, bicalutamide, bisantrene; bisnafide dimesylate, bizelesin, bleomycin sulfate, brequinar sodium, bropirimine, busulfan, cactinomycin, calusterone, caracemide, carbetimer, carboplatin, carmustine, carubicin, carzelesin, cedefingol, chlorambucil, cirolemycin, cladribine, crisnatol mesylate, cyclophosphamide, cytarabine, dacarbazine, daunorubicin, decitabine, dexormaplatin; dezaguanine, dezaguanine mesylate, diaziquone, doxorubicin, droloxifene, dromostanolone propionate, duazomycin, edatrexate, eflornithine, elsamitrucin, enloplatin, enpromate, epipropidine, epirubicin, erbulozole, esorubicin, estramustine, etanidazole, etoposide, etoposide, etoprine, fadrozole, fazarabine, fenretinide, floxuridine, fludarabine phosphate, fluorouracil, fluorocitabine, fosquidone, fostriecin sodium, gemcitabine, hydroxyurea, idarubicin, ifosfamide, ilmofosine, interleukin 11 (including recombinant interleukin 11, or rIL2), interferon alfa-2a, interferon alfa-2b, interferon alfa-n1, interferon alfa-n3, interferon beta-1a, interferon gamma-1b, iproplatin, irinotecan, lanreotide, letrozole, leuprolide, liarozole, lometrexol, lomustine, losoxantrone, masoprocol, maytansine, mechlorethamine, megestrol, melengestrol, melphalan, menogaril; mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nocodazole, nogalamycin, ormaplatin, oxisuran, pegaspargase, peliomycin, pentamustine, peplomycin, perfosfamide, pipobroman, piposulfan, piroxantrone, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tecogalan, tegafur, teloxantrone, temoporfin, teniposide, teroxirone, testolactone, thiamiprine, thioguanine, thiotepa, tiazofurin, tirapazamine, toremifene, trestolone, triciribine, trimetrexate; trimetrexate glucuronate, triptorelin, tubulozole, uracil mustard, uredepa, vapreotide, verteporfin, vinblastine, vincristine, vindesine, vinepidine, vinglycinate, vinleurosine, vinorelbine, vinrosidine, vinzolidine, vorozole, zeniplatin, zinostatin, zorubicin, 5-fluorouracil, and leucovorin.

31. The method according to claim 30, wherein the anti-cancer agent is TARCEVA (erlotinib).
